(19) United States
(12) Patent Application Publication
(10) Pub. No.: US 2026/0102327 A1
(43) Pub. Date: Apr. 16, 2026

(54) COSMETIC COMPOSITION FOR SKIN IMPROVEMENT CONTAINING LOW-MOLECULAR-WEIGHT COLLAGEN, COLLAGEN WATER, OR COLLAGEN EXTRACT

(71) Applicants: Sooy-K Co., Ltd., Seongnam-si (KR); BeautySelection Co.,Ltd, Seoul (KR)

(72) Inventors: Ji Sun CHUNG, Seongnam-si (KR); Youn Ok JUNG, Hwaseong-si (KR); Mi Hwa KIM, Gimpo-si (KR)

(73) Assignees: Sooy-K Co., Ltd., Seongnam-si (KR); BeautySelection Co.,Ltd, Seoul (KR)

(21) Appl. No.: 18/979,028

(22) Filed: Dec. 12, 2024

(30) Foreign Application Priority Data
Oct. 14, 2024 (KR) 10-2024-0139375

(51) Int. Cl. A61K 8/65; A61Q 19/00

(52) U.S. Cl. CPC A61K 8/65; A61Q 19/00

(57) ABSTRACT

The present invention relates to a cosmetic composition for skin improvement containing low-molecular-weight collagen, collagen water, or a collagen extract. According to one embodiment of the technical idea of the present invention, a cosmetic composition for skin improvement containing low-molecular-weight collagen, collagen water, or a collagen extract includes one or more collagens selected from low-molecular-weight collagen, collagen water or a collagen extract, and the collagen powder is a collagen powder having a molecular weight of 200 to 300 daltons (Da). According to various embodiments of the technical idea of the present invention, by the above-described configuration, a cosmetic composition for skin improvement containing low-molecular-weight collagen, collagen water, or a collagen extract can have excellent skin soothing, skin moisturizing, and skin barrier improvement effects and improve skin health using low-molecular-weight collagen, collagen water, or a collagen extract as a cosmetic composition.

### CROSS-REFERENCE TO RELATED APPLICATION

This application claims priority to and the benefit of Korean Patent Application No. 10-2024-0139375, filed on Oct. 14, 2024, the disclosure of which is incorporated herein by reference in its entirety.

### BACKGROUND

### 1. Field of the Invention

The present invention relates to a cosmetic composition for skin improvement containing low-molecular-weight collagen, collagen water, or a collagen extract, and more specifically, to a cosmetic composition for skin improvement containing low-molecular-weight collagen, collagen water, or a collagen extract, which has excellent skin soothing, skin moisturizing, and skin barrier improvement effects and can improve skin health using low-molecular-weight collagen, collagen water, or a collagen extract as a cosmetic composition.

### 2. Discussion of Related Art

In modern society, a large amount of reactive oxygen is generated due to exhaust gases, harmful chemicals, ultraviolet rays resulting from destruction of the ozone layer, water pollution, and the like, and such reactive oxygen penetrates the human body and causes various side effects, and is known to be the main cause of pigment changes and skin aging.

In addition, in recent years, in order to relieve excessive social and work-related stress, outdoor leisure activities such as golf are increasing, and during these outdoor leisure activities, since the skin of the body is more easily directly exposed to the pollutants, skin damage and skin aging cannot be avoided, which has a negative effect on the trend of enjoying outdoor leisure activities more actively and pleasantly.

In general, the skin has a primary function of protecting us from various external factors that disrupt our body's homeostasis through basic metabolic processes. That is, the skin is located on the outermost part of the body and performs various roles such as protection through physical, chemical, and biological barrier functions, temperature regulation, sensory function, secretion and excretion, absorption, storage, regeneration, and the like, but it is most easily damaged when continuous external stress is applied.

Exposure to ultraviolet rays, exhaust gases, yellow dust, environmental pollutants, heavy metals, temperature changes, and microorganisms are representative examples of the main cause of external stress, and they mainly generate reactive oxygen species (ROS) through oxidative stress, which causes the antioxidant defense system to collapse and lead to cell damage and death.

Conventionally, there are cosmetics that prevent skin aging by providing elasticity to the skin and inhibiting the development of wrinkles, such as cosmetics that contain moisturizing components such as collagen, elastin, natural moisturizing factors, and glycerin, and cosmetics that contain vitamins such as vitamin A, vitamin C, vitamin E and derivatives, and cell regeneration components such as licorice acid extracted from plants, beta-carotene, and amino acids involved in signaling to fibroblasts.

However, when exposed to ultraviolet rays or air for a long period of time, these conventional cosmetics have various problems in that not only the raw materials used in them are precipitated or discolored due to recrystallization but also it is difficult to maintain potency, resulting in insufficient efficacy or causing side effects on the skin.

In addition, recently, the number of people with skin in which the stratum corneum cannot perform its normal skin barrier function is increasing due to the increase in harmful factors for the skin, the slowing down of the rate of production and shedding of the skin stratum corneum due to changes in eating habits, and the reduction in the amount of moisturizing factors and lipids in the stratum corneum due to the decrease in the function of keratinocytes.

This breakdown of the skin barrier function causes various skin diseases such as dry skin, atopic dermatitis, contact dermatitis, and psoriasis. Although symptoms of these diseases can be expected to be alleviated with just conventional moisturizers that have moisture retention functions, it is difficult to achieve a fundamental cure.

Accordingly, the inventors of the present invention have completed the present invention by preparing a cosmetic composition containing low-molecular-weight collagen, collagen water, or a collagen extract and confirming that the composition has excellent skin soothing, skin moisturizing, and skin barrier improvement effects and can improve skin health.

### RELATED ART DOCUMENTS

### Patent Documents

(Patent Document 01) Korean Registered Patent No. 10-2139471 (registered on Jul. 24, 2020)
(Patent Document 02) Korean Laid-Open Patent Publication No. 10-2023-0120546 (published on Aug. 17, 2023)
(Patent Document 03) Korean Laid-Open Patent Publication No. 10-2023-0119873 (published on Aug. 16, 2023)

### SUMMARY OF THE INVENTION

The present invention is directed to providing a cosmetic composition for skin improvement containing low-molecular-weight collagen, collagen water, or a collagen extract, which has excellent skin soothing, skin moisturizing, and skin barrier improvement effects and can improve skin health using low-molecular-weight collagen, collagen water, or a collagen extract as a cosmetic composition

In addition, the present invention is directed to providing a cosmetic composition for skin improvement containing low-molecular-weight collagen, collagen water, or a collagen extract, which can cleanly remove not only makeup but also dead skin cells and pore impurities from the skin, minimize skin troubles and skin irritation, and moisturize and smooth parched and dry skin.

The various objectives to be accomplished by the present invention are not limited to the objectives mentioned above, and other objectives not mentioned will be clearly understood by those skilled in the art from the following description.

In one embodiment of the technical idea of the present invention, a cosmetic composition for skin improvement containing low-molecular-weight collagen, collagen water, or a collagen extract is disclosed.

The cosmetic composition for skin improvement containing the low-molecular-weight collagen, collagen water, or collagen extract includes one or more collagens selected from low-molecular-weight collagen, collagen water or a collagen extract, and the collagen powder is a collagen powder having a molecular weight of 200 to 300 daltons (Da).

The low-molecular-weight collagen powder may be collagen powder which is prepared by preparing one or more collagens selected from the group consisting of collagen extracted from fish and fish scales, chicken foot collagen, beef foot collagen, and pork foot collagen, sterilizing the collagen, mixing 80 to 120 parts by weight of the sterilized collagen, 20 to 40 parts by weight of a proteolytic enzyme, and 150 to 250 parts by weight of purified water to produce a collagen enzyme mixture, heating the collagen enzyme mixture in a double boiler at a temperature of 57 to 635° C. for 1 to 5 hours to produce a collagen enzyme hydrolyzate, immersing the collagen enzyme hydrolyzate in sodium hypochlorite for 10 to 60 minutes to decolorize and deodorize it, concentrating the decolorized and deodorized collagen enzyme hydrolyzate to produce a collagen concentrate, and spray drying the collagen concentrate at a temperature of 160 to 180° C.

The collagen water may be collagen water which is prepared by preparing one or more collagens selected from the group consisting of collagen extracted from fish and fish scales, chicken foot collagen, beef foot collagen, and pork foot collagen, sterilizing the collagen, adding 10- to 30-fold (v/w) distilled water to the sterilized collagen, heating the mixture at 60 to 80° C. for 10 to 50 minutes, then distilling and extracting the mixture while heating at 100° C. to recover the distillate at 30 to 40% of the total weight of the mixture and produce a collagen distillation extract, and filtering the collagen distillation extract through a filter.

The collagen extract may be a collagen extract which is prepared by preparing one or more collagens selected from the group consisting of collagen extracted from fish and fish scales, chicken foot collagen, beef foot collagen, and pork foot collagen, sterilizing the collagen, mixing 80 to 120 parts by weight of purified water based on 100 parts by weight of the total content of the sterilized collagen, heating the mixture at a temperature of 90 to 100° C. for 1 to 3 hours and performing hot water extraction to produce a collagen hot water extract, and filtering the collagen hot water extract through a filter.

In addition to the low-molecular-weight collagen powder, collagen water, and collagen extract, the cosmetic composition may include caffeic acid, soybean-derived lysolecithin, a fermented herbal extract, coenzyme Q10, tocopherol acetate, butylene glycol, stearyl trimethyl ammonium chloride, L-aspartic acid, sorbitol, stearyl alcohol, cyclopentasiloxane, vitamin C, madecassoside, olive liquid, sodium laureth sulfate, cocamide MIPA, brewer's yeast extract, an acai berry extract, emollient oil, Artemisiae capillaris extract, Allium hookeri extract, grapefruit seed extract, sodium alginate, Moutan cortex radicis extract, pine needle shoot extract, panthenol, salicylic acid, polyquaternium-7, niacinamide, jojoba seed oil, behenyl alcohol, myristyl alcohol, cyclodextrin, a fragrance, and the balance as deionized water.

The cosmetic composition may include 0.1 to 0.3 wt % of the low-molecular-weight collagen powder, 0.5 to 1.5 wt % of the collagen water, 0.1 to 1 wt % of the collagen extract, 0.1 to 1 wt % of caffeic acid, 0.1 to 0.5 wt % of soybean-derived lysolecithin, 0.5 to 1.5 wt % of fermented herbal extract, 0.08 to 0.12 wt % of coenzyme Q10, 0.1 to 0.3 wt % of tocopherol acetate, 0.5 to 1.5 wt % of butylene glycol, 0.1 to 0.3 wt % of stearyl trimethyl ammonium chloride, 0.08 to 0.12 wt % of L-aspartic acid, 0.05 to 0.15 wt % of sorbitol, 0.5 to 1.5 wt % of stearyl alcohol, 0.8 to 1.2 wt % of cyclopentasiloxane, 0.1 to 0.5 wt % of vitamin C, 0.05 to 0.15 wt % of madecassoside, 0.01 to 0.1 wt % of olive liquid, 0.8 to 1.2 wt % of sodium laureth sulfate, 0.1 to 0.5 wt % of cocamide MIPA, 0.05 to 0.15 wt % of brewer's yeast extract, 0.01 to 0.1 wt % of acai berry extract, 0.08 to 0.12 wt % of emollient oil, 0.01 to 0.1 wt % of Artemisiae capillaris extract, 0.01 to 0.05 wt % of Allium hookeri extract, 0.01 to 0.05 wt % of grapefruit seed extract, 0.01 wt % to 0.05 wt % of sodium alginate, 0.01 to 0.05 wt % of Moutan cortex radicis extract, 0.01 to 0.05 wt % of pine needle shoot extract, 0.04 to 0.08 wt % of panthenol, 0.01 to 0.05 wt % of salicylic acid, 0.08 to 0.12 wt % of polyquaternium-7, 0.07 to 0.13 wt % of niacinamide, 0.1 to 0.3 wt % of jojoba seed oil, 0.1 to 1 wt % of behenyl alcohol, 0.5 to 1.5 wt % of myristyl alcohol, 0.07 to 0.13 wt % of cyclodextrin, 0.01 to 0.1 wt % of fragrance, and the balance as deionized water.

Specific details of other embodiments are included in the detailed description.

### Advantageous Effects

According to various embodiments of the technical idea of the present invention, the cosmetic composition for skin improvement containing low-molecular-weight collagen, collagen water, or a collagen extract can have excellent skin soothing, skin moisturizing, and skin barrier improvement effects and improve skin health using low-molecular-weight collagen, collagen water, or collagen extract as a cosmetic composition.

In addition, the cosmetic composition for skin improvement containing low-molecular-weight collagen, collagen water, or a collagen extract according to various embodiments of the technical idea of the present invention can cleanly remove not only makeup but also dead skin cells and pore impurities from the skin, minimize skin troubles and skin irritation, and moisturize and smooth parched and dry skin.

It will be fully appreciated that various embodiments of the technical idea of the present invention can provide various effects not specifically mentioned.

### BRIEF DESCRIPTION OF THE DRAWINGS

The above and other objects, features and advantages of the present invention will become more apparent to those of ordinary skill in the art by describing exemplary embodiments thereof in detail with reference to the accompanying drawings, in which:

FIG. 1 is a graph showing the skin brightness improvement effect of cosmetics according to Examples 1, 2, and 3 and cosmetics according to Comparative Examples 1 and 2; and

FIG. 2 is a graph showing the skin wrinkle improvement effect of cosmetics according to Examples 1, 2, and 3 and cosmetics according to Comparative Examples 1 and 2.

### DETAILED DESCRIPTION OF EXEMPLARY EMBODIMENTS

Advantages and features of the present invention and methods of achieving them will become apparent with reference to the embodiments described in detail below. However, the present invention is not limited to the embodiments described herein, and may be embodied in other forms. Rather, the embodiments introduced herein are provided so that the disclosure will be thorough and complete, and so that the spirit of the present invention may be sufficiently conveyed to those skilled in the art.

The terms used in the present application are only used to describe specific embodiments, and are not intended to limit the present invention. Singular expressions include plural expressions unless the context clearly dictates otherwise.

Unless defined otherwise, all terms used herein, including technical or scientific terms, have the same meaning as commonly understood by one of ordinary skill in the art to which the present invention belongs. Terms such as those defined in a commonly used dictionary should be interpreted as having a meaning consistent with the meaning in the context of the related art, and should not be interpreted in an ideal or excessively formal meaning unless explicitly defined in the present application.

Hereinafter, a cosmetic composition for skin improvement containing low-molecular-weight collagen, collagen water, or a collagen extract according to one embodiment of the technical idea of the present invention will be described in more detail through exemplary embodiments.

According to one embodiment of the technical idea of the present invention, a cosmetic composition for skin improvement containing low-molecular-weight collagen, collagen water, or a collagen extract may include one or more collagens selected from low-molecular-weight collagen, collagen water or a collagen extract.

The collagen is a structural protein found in most animals, and particularly, it constitutes the flesh and connective tissue of mammals. The human body contains about 20% protein, of which collagen accounts for 30% (L. C. Junqueira and J. Carneiro 1983), and the collagen is also found in large quantities in the bones and skin of cows, pigs, chickens, and fish. Currently, this collagen is used in various products ranging from health foods to cosmetics and pharmaceuticals.

The collagen may be divided into high-molecular-weight collagen with a molecular weight of 5,000 daltons or more and low-molecular-weight collagen with a molecular weight of 1,500 daltons or less, depending on the molecular weight. Low-molecular-weight collagen is known to have a relatively higher absorption rate in the body than high-molecular-weight collagen, so it is used in health functional foods, cosmetics, and general medicines.

Collagen, the main protein that constitutes the skin, is a fibrous protein that accounts for about 90% of the dermis, and along with elastin, collagen provides skin tension and elasticity and has a function of protecting the skin from ultraviolet rays and preventing wrinkles. It is known that collagen should have a molecular weight of at least 500 Da to be delivered to the dermis through the epidermis.

In muscle tissue, collagen acts as a major component of the endomysium. Collagen has a molecular weight of 300,000, and three α-chains each having a molecular weight of 100,000 come together to form a γ-chain, creating a right-handed spiral that rotates once every 40 residues. Collagen has a triple helix structure with a diameter of 15 nm and a length of 300 nm. The a chains are strongly bound by some hydrogen bonding, and when microfibrils gather, they become collagen fibrils through covalent cross-linking, and these gather to form fibers.

The low-molecular-weight collagen powder may be a collagen powder having a molecular weight of 200 to 300 daltons (Da), and the low-molecular-weight collagen powder may be a collagen powder prepared by the following method.

First, collagen may be prepared to produce low-molecular-weight collagen powder.

As the collagen, various kinds of animals known to contain collagen may be used. For example, as the animal collagen-containing material, one or more collagens selected from the group consisting of collagen extracted from fish and fish scales, chicken foot collagen, beef foot collagen, and pork foot collagen may be used.

Next, the collagen may be sterilized.

The above sterilization may utilize a known sterilization method such as ultra-high temperature (UHT) sterilization or high temperature short time (HTST) sterilization. Specifically, the sterilization conditions using the high temperature short time (HTST) sterilization may be sterilization at a temperature of 70 to 75° C. for 10 to 20 seconds, and the sterilization conditions using the ultra-high temperature (UHT) sterilization may be sterilization at a temperature of 130 to 140° C. for 2 to 10 seconds.

Next, the sterilized collagen may be enzymatically decomposed to produce a collagen enzyme hydrolyzate.

The enzymatic decomposition may be performed by mixing 80 to 120 parts by weight of the sterilized collagen, 20 to 40 parts by weight of a proteolytic enzyme, and 150 to 250 parts by weight of purified water to prepare a collagen enzyme mixture, and heating the collagen enzyme mixture in a double boiler at a temperature of 57 to 635° C. for 1 to 5 hours. For example, one or more selected from the group consisting of aminopeptidase, Flavourzyme, Protamax, a protease, Neutrase, and Alcalase may be used as the proteolytic enzyme.

Specifically, as the proteolytic enzyme, a commercially available product may be purchased and used. For example, the proteolytic enzyme may be used by mixing Alcalase (Novozymes, Bagsvaerd, Denmark) and Protease-NP (Bioland, Asan, Korea) in a weight ratio of 1:1.

Next, the collagen enzyme hydrolyzate may be decolorized and deodorized.

By decolorizing and deodorizing the collagen enzyme hydrolyzate, a collagen enzyme hydrolyzate with no odor and bleached white may be prepared. For example, the decolorization and deodorization may be performed by immersing the collagen enzyme hydrolyzate in sodium hypochlorite for 10 to 60 minutes to decolorize and deodorize it.

Next, the decolorized and deodorized collagen enzyme hydrolyzate may be concentrated to produce a collagen concentrate.

The collagen concentrate may be prepared by concentrating the decolorized and deodorized collagen enzyme hydrolyzate using a known concentrator. The configuration for concentrating using the concentrator is a known technology, so a detailed description thereof will be omitted for the convenience of explanation and clarity of the technical idea of the present invention.

Next, the collagen concentrate may be dried to produce a collagen powder having a molecular weight of 200 to 300 daltons (Da).

For example, the collagen powder may be prepared by spray drying the collagen concentrate at a temperature of 160 to 180° C.

In addition, the collagen water may be collagen water prepared by the following method.

First, in order to produce the collagen water, collagen may be prepared.

As the collagen, various kinds of animals known to contain collagen may be used. For example, as the animal collagen-containing material, one or more collagens selected from the group consisting of collagen extracted from fish and fish scales, chicken foot collagen, beef foot collagen, and pork foot collagen may be used.

Next, the collagen may be sterilized.

The above sterilization may utilize a known sterilization method such as ultra-high temperature (UHT) sterilization or high temperature short time (HTST) sterilization. Specifically, the sterilization conditions using the high temperature short time (HTST) sterilization may be sterilization at a temperature of 70 to 75° C. for 10 to 20 seconds, and the sterilization conditions using the ultra-high temperature (UHT) sterilization may be sterilization at a temperature of 130 to 140° C. for 2 to 10 seconds.

Next, the sterilized collagen may be distilled and extracted to produce a collagen distillation extract.

For example, the collagen distillation extract may be prepared by adding 10- to 30-fold (v/w) distilled water to the sterilized collagen, heating the mixture at 60 to 80° C. for 10 to 50 minutes, and then distilling and extracting the mixture while heating at 100° C. to recover the distillate at 30 to 40% of the total weight of the mixture.

Next, the collagen distillation extract may be filtered through a filter to produce collagen water.

For example, the collagen water may be prepared by filtering the collagen distillation extract through a known filter to remove solids, thereby extracting useful components of collagen.

In addition, the collagen extract may be a collagen extract prepared by the following method.

First, to produce the collagen extract, collagen may be prepared.

As the collagen, various kinds of animals known to contain collagen may be used. For example, as the animal collagen-containing material, one or more collagens selected from the group consisting of collagen extracted from fish and fish scales, chicken foot collagen, beef foot collagen, and pork foot collagen may be used.

Next, the collagen may be sterilized.

The above sterilization may utilize a known sterilization method such as ultra-high temperature (UHT) sterilization or high temperature short time (HTST) sterilization. Specifically, the sterilization conditions using the high temperature short time (HTST) sterilization may be sterilization at a temperature of 70 to 75° C. for 10 to 20 seconds, and the sterilization conditions using the ultra-high temperature (UHT) sterilization may be sterilization at a temperature of 130 to 140° C. for 2 to 10 seconds.

Next, the sterilized collagen may be extracted with hot water to produce a collagen hot water extract.

For example, the collagen hot water extract may be prepared by mixing 80 to 120 parts by weight of purified water based on 100 parts by weight of the total content of the sterilized collagen, then heating the mixture at a temperature of 90 to 100° C. for 1 to 3 hours and performing hot water extraction.

Next, the collagen hot water extract may be filtered through a filter to produce a collagen extract.

For example, the collagen extract may be prepared by filtering the collagen hot water extract through a known filter to remove solids, thereby extracting useful components of collagen.

According to one embodiment of the technical idea of the present invention, the cosmetic composition for skin improvement containing low-molecular-weight collagen, collagen water, or a collagen extract includes caffeic acid, soybean-derived lysolecithin, a fermented herbal extract, coenzyme Q10, tocopherol acetate, butylene glycol, stearyl trimethyl ammonium chloride, L-aspartic acid, sorbitol, stearyl alcohol, cyclopentasiloxane, vitamin C, madecassoside, olive liquid, sodium laureth sulfate, cocamide MIPA, brewer's yeast extract, acai berry extract, emollient oil, Artemisiae capillaris extract, Allium hookeri extract, grapefruit seed extract, sodium alginate, Moutan cortex radicis extract, pine needle shoot extract, panthenol, salicylic acid, polyquaternium-7, niacinamide, jojoba seed oil, behenyl alcohol, myristyl alcohol, cyclodextrin, a fragrance, and the balance as deionized water.

Specifically, according to one embodiment of the technical idea of the present invention, the cosmetic composition for skin improvement containing low-molecular-weight collagen, collagen water, or a collagen extract may include 0.1 to 0.3 wt % of the low-molecular-weight collagen powder, 0.5 to 1.5 wt % of the collagen water, 0.1 to 1 wt % of the collagen extract, 0.1 to 1 wt % of caffeic acid, 0.1 to 0.5 wt % of soybean-derived lysolecithin, 0.5 to 1.5 wt % of fermented herbal extract, 0.08 to 0.12 wt % of coenzyme Q10, 0.1 to 0.3 wt % of tocopherol acetate, 0.5 to 1.5 wt % of butylene glycol, 0.1 to 0.3 wt % of stearyltrimethylammonium chloride, 0.08 to 0.12 wt % of L-aspartic acid, 0.05 to 0.15 wt % of sorbitol, 0.5 to 1.5 wt % of stearyl alcohol, 0.8 to 1.2 wt % of cyclopentasiloxane, 0.1 to 0.5 wt % of vitamin C, 0.05 to 0.15 wt % of madecassoside, 0.01 to 0.1 wt % of olive liquid, 0.8 to 1.2 wt % of sodium laureth sulfate, 0.1 to 0.5 wt % of cocamide MIPA, 0.05 to 0.15 wt % of brewer's yeast extract, 0.01 to 0.1 wt % of acai berry extract, 0.08 to 0.12 wt % of emollient oil, 0.01 to 0.1 wt % of Artemisiae capillaris extract, 0.01 to 0.05 wt % of Allium hookeri extract, 0.01 to 0.05 wt % of grapefruit seed extract, 0.01 wt % to 0.05 wt % of sodium alginate, 0.01 to 0.05 wt % of Moutan cortex radicis extract, 0.01 to 0.05 wt % of pine needle shoot extract, 0.04 to 0.08 wt % of panthenol, 0.01 to 0.05 wt % of salicylic acid, 0.08 to 0.12 wt % of polyquaternium-7, 0.07 to 0.13 wt % of niacinamide, 0.1 to 0.3 wt % of jojoba seed oil, 0.1 to 1 wt % of behenyl alcohol, 0.5 to 1.5 wt % of myristyl alcohol, 0.07 to 0.13 wt % of cyclodextrin, 0.01 to 0.1 wt % of fragrance, and the balance as deionized water.

The caffeic acid is an organic compound classified as hydroxycinnamic acid and is composed of a phenol group and an acrylic group. Caffeic acid is found in all plants because this compound is a key intermediate in the biosynthesis of lignin, a major source of biomass. Caffeic acid has been shown to have potent antioxidant activity in vitro and in vivo, and also possesses immunomodulatory and anti-inflammatory activities.

In the present invention, the caffeic acid may be a known caffeic acid. For example, a caffeic acid derivative represented by the following Chemical Formula 1 may be used as the caffeic acid.

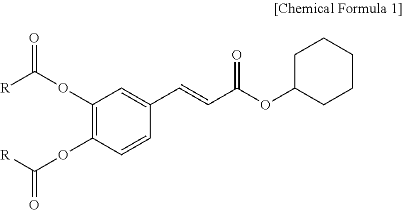

In Chemical Formula 1, R is a C₄₋₆ side chain alkyl or cyclohexyl.

Specifically, the caffeic acid derivative represented by Chemical Formula 1 may be one or more selected from (E)-4-(3-(cyclohexyloxy)-3-oxoprop-1-en-1-yl)-1,2-phenylene bis(2,2-dimethylpropanoate) or (E)-4-(3-(cyclohexyloxy)-3-oxoprop-1-en-1-yl)-1,2-phenylene dicyclohexanecarboxylate.

The soybean-derived lysolecithin is lysolecithin extracted from soybeans, and the lysolecithin functions as a percutaneous penetration enhancer and emulsifier and may contribute to enhancing skin absorption.

The lysolecithin may be a known lysolecithin extracted from soybeans. Soybeans, one of our country's traditional foods, are generally used as a source of high-quality protein and lipids, and the soybeans are used in a variety of ways, from fermented foods such as soybean paste and soy sauce to non-fermented foods such as tofu, bean sprouts, and soybean powder. Recently, soybeans have begun to receive attention as a functional food that can prevent adult diseases, including aging and skin whitening.

Up to now, much research has been conducted on the skin aging, skin whitening, and skin moisturizing effects of soybeans, and the number of skin beauty products using soybeans is also increasing. Korean Laid-Open Patent Publication No. 10-2004-0106893 discloses a Glycine Semen germinatum extract and a functional cosmetic for preventing skin aging containing the same as an active ingredient, and Korean Laid-Open Patent Publication No. 10-2005-0079478 discloses a rich soybean paste extract obtained by fermenting soybeans with Bacillus bacteria and a cosmetic composition containing the same. An invention relating to a cosmetic composition using soybeans includes a fermented soybean extract obtained by fermenting soybeans with Bacillus bacteria and a cosmetic composition containing the same (Korean Registered Patent No. 10-0810006).

In the present invention, the lysolecithin may be lysolecithin prepared by extraction from soybeans. The composition of the lysolecithin is a known technology, so a detailed description thereof will be omitted for the convenience of explanation and clarity of the technical idea of the present invention.

The fermented herbal extract may be prepared by fermenting and then extracting herbal materials, and the fermented herbal extract may be prepared by the following method.

That is, in order to produce the fermented herbal extract, herbal materials may first be prepared.

The herbal materials may include peony, mulberry leaf, chinaroot, balloon flower, ginger, licorice, macadamia, black cumin seed, chia seed, Moringa seed, and safflower seed, and the herbal materials may include 10 to 20 parts by weight of peony, 1 to 10 parts by weight of mulberry leaf, 5 to 10 parts by weight of chinaroot, 5 to 15 parts by weight of balloon flower, 10 to 20 parts by weight of ginger, 1 to 3 parts by weight of licorice, 1 to 5 parts by weight of macadamia, 1 to 5 parts by weight of black cumin seed, 1 to 5 parts by weight of chia seed, 1 to 5 parts by weight of Moringa seed, and 1 to 5 parts by weight of safflower seed.

The peony (Paeonia lactiflora Pall) belongs to the peony family and is classified into red peony and white peony. Peony is known to be widely distributed in a wide range of areas, including the northeastern region of China north of Jiangsu Province, Mongolia, southern Far East Asia, and Korea. Peony contains a large amount of various sugars, mucilage, organic acids, and trace minerals, and is known to treat nutritional deficiencies and have an astringent action. In addition, peony improves vascular function, strengthens the digestive system, and suppresses pain in the nervous system. In addition, peony is known to suppress gastric acid secretion, strengthen bladder movements, relieve abdominal pain, and have excellent antibacterial, anti-inflammatory, and antipyretic actions.

The mulberry leaf is a leaf of a plant belonging to the genus Morus in the family Moraceae, and has been used as feed for silkworms for thousands of years. However, mulberry leaf has also been used as a herbal medicine material in folk remedies, and recently, has been receiving attention as a functional food as research on nutrients and physiologically active components useful for the human body is being conducted.

The mulberry leaves contain moisture, carbohydrates, proteins (25-35%), and 25 kinds of amino acids, of which serine and tyrosine, which are helpful for preventing cerebral blood circulation and senile dementia, are contained at 1.2% and 0.8%, respectively, and the lipid content is approximately 35%, with most fatty acids being unsaturated fatty acids and also contain 0.08% plant sterols, which are known to reduce blood cholesterol levels.

In addition, it contains a lot of glutamic acid and aspartic acid, and much more calcium, iron, potassium, and dietary fiber among minerals compared to green tea. The physiological activities of mulberry leaves include blood lipid suppression, blood sugar enhancement, heavy metal adsorption inhibition, detoxification, and antioxidant effects. Components found to be physiologically active include GABA, which lowers blood pressure, rutin, which strengthens capillaries and especially prevents bleeding in the brain, and flavonoid compounds that have excellent antioxidant, diuretic, and anticapillary permeability effects.

The chinaroot refers to the dried rhizome of the Smilax china L., a plant of the Smilax genus. Smilax china L. grows on all mountainsides south of the central part of Korea, and is dug up in the fall or spring, washed with water, cut into appropriate lengths, and dried in the sun to be used as a medicinal herb. Chinaroot is known to act on the stomach and liver meridian, reduce fever, eliminate dampness, and detoxify. It has also been reported to be used for joint pain, syphilis, scrofula, inflamed area, malignant abscesses, and mercury poisoning.

The physiological activities of the chinaroot have been reported to have a wide variety of pharmacological actions, including promotion of growth hormone secretion (Korean Registered Patent No. 10-0521845 and Korean Registered Patent No. 10-0498277), treatment of inflammatory diseases (Korean Registered Patent No. 10-1093730), prevention of vascular diseases (Korean Registered Patent No. 10-1156636), skin whitening (Korean Registered Patent No. 10-1086669), wrinkle improvement and skin damage relief (Korean Registered Patent No. 10-1086225), an effect of reducing heavy metals in the body (Korean Journal of Food and Nutrition, Vol. 24, No. 2, 233-238, 2011), antioxidant and antibacterial activities of Smilax china L. leaf extract (Korean Journal of Food Preservation, Vol. 18, No. 5, 764-772, 2011), an antifungal action (Korean J. of pharmacology, Vol. 9, No. 2, 1973), protection of brain nerves or prevention of degenerative brain diseases (Korean Registered Patent No. 10-0779179), and prevention or treatment of obesity, hyperlipidemia or fatty liver (Korean Registered Patent No. 10-1225486).

The balloon flower (Platycodon grandiflorum) grows widely in mountainous regions of Korea, Japan, and China, and is a wild vegetable food that is used as a medicinal herb in oriental medicine and is also widely consumed as a general food and contains triterpenoid saponins, glucides, and fiber. These saponins have been shown to have antitussive, expectorant, central nervous system suppression, anti-acute and chronic inflammation, anti-ulcer, and gastric juice secretion suppression effects, a blood sugar-lowering effect by expanding blood vessels, a cholesterol metabolism improvement effect, and antioxidant and anti-cancer effects in animal experiments, and the number of traditional Korean medicine prescriptions containing balloon flower as a herbal medicine is 287 in Donguibogam and 49 in Bangyakhappyeon, showing that it has diverse pharmacological actions.

The balloon flower is not only good for coughs, colds, and asthma, but also suppresses melanin pigment production to make the skin white and smooth, and it also helps to discharge waste products, promote blood circulation, and has anti-inflammatory effects, making it effective in treating acne.

In addition, balloon flower roots contain about 2% of saponin, and the balloon flower saponin has a hemolytic action, relieves coughs and phlegm, reduces inflammation, has a blood sugar lowering action, and lowers cholesterol. In addition, they contain a lot of calcium, which is good for preventing osteoporosis and maintaining bone health in growing children.

The ginger (Zingiber officinale) is a perennial plant belonging to the genus Zingiber in the family Zingiberaceae, its rhizome is used as a herbal medicinal, and has a spicy and bitter taste. The aroma and spicy taste of ginger are known to be due to a component called gingerol, which is known to have excellent antioxidant and anti-inflammatory properties.

The licorice (Glycyrrhiza uralensis) is a perennial grass belonging to the legume family, its reddish-brown roots are sweet and are used in various ways, such as for food and medicinal sweeteners. Its main components include triterpene saponins and flavonoids, and it is known to exhibit various physiological activities such as anti-inflammation, antibacterial, antioxidant, antithrombotic, antidiabetic, and antiviral activities.

The macadamia nuts consist of 75% natural edible oil, 9% protein, 9% carbohydrates and 2% dietary fiber. The remainder contains calcium, phosphorus, sodium, vitamins E, B1, B2, niacin, and iron, which are essential for human nutrition, and also contains selenium, which is an essential trace element in the body that has been shown to delay the onset of AIDS and promote growth in children in addition to having anti-cancer effects.

The vitamins and unsaturated fatty acids contained in the macadamia add moisture to dry skin and help blood circulation, thereby increasing the vitality of skin cells. In addition, it contains unsaturated fatty acids, proteins, and minerals, which protect the skin from reactive oxygen and have an anti-aging effect, helping maintain smooth and healthy skin.

The black cumin seed (Citrus Junos seed) is called the black seed of Nigella sativa. It is a plant belonging to the Ranunculaceae family, which is a unique plant that has been used as a medicinal herb for over 2,000 years in the Middle East, Southwest Asia, or Southeast Asia, and in fact, it is composed of components such as thymoquinone, thymohydroquinone, nigellimine-N-oxide, nigellicine, nigellidine, nigellone, dithymoquinone, thymol, carvacrol, 6-methoxycoumarin, 7-hydroxy-coumarin, oxycoumarin, alpha-hedrin, sterylglucoside, tannins, flavinoids, essential fatty acids, essential amino acids, ascorbic acid, iron, and calcium.

The thymoquinone component is particularly effective in treating fungi and is also used to treat athlete's foot, fungal infections, and fungal vaginitis. It is also known to have the effect of alleviating psoriasis and acne, and in the medical field, it is known to help improve immune diseases, asthma, and allergies, and is also used for various inflammations and skin diseases such as eczema.

The chia seeds are rich in omega-3 fatty acids and are a treasure trove of nutrients, including iron, potassium, calcium, magnesium, and dietary fiber. They are called the dream food for dieters because they provide a feeling of fullness, make people eat less of other foods, and are composed of soluble fiber, which helps remove waste from the intestines.

That is, chia seeds are known to be the food with the highest content of omega-3 fatty acids, have more calcium than milk, have twice as much potassium as bananas, and have three times more iron than spinach.

These chia seeds have the gelatin property of turning into jelly when placed in water and expanding about 10-fold in volume, making them filling, and are particularly rich in nutrients, making them effective in consuming nutrients that may be lacking due to dieting, and they are preferred as a diet food because they break down body fat and prevent fat from forming, and they are rich in dietary fiber, which helps improve constipation by activating the intestines and promoting effective bowel movements, and they are also good for antioxidant actions, anti-aging, and skin beauty, and improving blood circulation, making them a food with many effects.

The Moringa seed is the seed of the Moringa tree, which is a plant of the Moringaceae family and the Moringa genus, and the scientific name thereof is Moringa oleifera (aka Moringa pterygosperma), and in Tamil, from which the scientific name is derived, it is also called Murunggai. Moringa is a legume and tropical tree whose leaves and fruits and even the entire tree are edible, and contains over 90 nutrients, including a large amount of amino acids, minerals, and vitamins.

In particular, the water-soluble proteins contained in Moringa seeds play a role in purifying contaminants in water. When these water-soluble proteins are applied to the skin, they exhibit a detoxifying effect that purifies the skin of impurities and leaves the skin clean. In addition, the water-soluble proteins contain a large amount of carotenoids such as beta-carotene and rutin, as well as vitamins and minerals, which can brighten dull skin and provide a whitening effect.

The safflower seed contains linoleic acid, which is an unsaturated fatty acid, and inorganic materials, which are mineral elements such as potassium, calcium, magnesium, gold, aluminum, bromine, copper, iron, manganese, rubidium, and zinc, as well as carthamine and chalconic acid glycosides. In particular, safflower sprouts contain a large amount of luteolin, an antioxidant, and thus it has been found to have a medicinal effect that promotes metabolism in the body and treats various diseases, which has led to an explosive increase in consumption.

Next, fermented herbal medicine materials may be prepared by mixing a microbial culture solution with the prepared herbal medicine materials and then fermenting the mixture.

For example, the fermented herbal materials may be prepared by mixing 10 to 30 parts by weight of a microbial culture solution based on 100 parts by weight of the total content of the prepared herbal medicine materials, and then fermenting the herbal medicine material mixture mixed with the microbial culture solution at a temperature of 38 to 42° C. for 10 to 20 days.

The microbial culture solution may be a microbial culture solution prepared by the following method.

For example, to produce the microbial culture solution, first, a medium may be prepared and then sterilized.

As the medium, a general medium used for culturing microorganisms may be prepared, and the process may be performed by sterilizing the prepared medium at a temperature of 80 to 90° C. for 40 to 80 minutes and then cooling it at a temperature of 30 to 35° C.

The medium used to cultivate the microorganism has a known composition, so a detailed description thereof will be omitted for the convenience of explanation and clarity of the technical idea of the present invention.

Next, a culture medium may be prepared by mixing a culture solution, which includes nutrients necessary for culturing microorganisms, into the medium.

The culture medium may be prepared by mixing a culture solution into a medium, for example, the culture solution includes glucose, dextrin, glycerin, carrageenan (C₂₄H₃₆O₂₅S₂), lysine (C₆H₁₄N₂O₂), potassium chloride (KCl), sodium citrate, ethylene glycol (CH₂OHCH₂OH), magnesium chloride, peptone, malt extract, and pyridoxine hydrochloride (C₉H₁₁NO₃HCl).

Specifically, the culture solution may include, per 1 liter (L) of purified water, 65 g/L of glucose, 25 g/L of dextrin, 20 g/L of glycerin, 20 g/L of carrageenan (C₂₄H₃₆O₂₅S₂), 15 g/L of lysine (C₆H₁₄N₂O₂), 13 g/L of potassium chloride (KCl), 8 g/L of sodium citrate, 8 g/L of ethylene glycol (CH₂OHCH₂OH), 3 g/L of magnesium chloride, 5 g/L of peptone, 5 g/L of malt extract, and 1 g/L of pyridoxine hydrochloride (C₉H₁₁NO₃HCl), and the culture medium may be prepared by mixing 80 to 120 parts by weight of the culture solution based on 100 parts by weight of the total content of the medium.

The carrageenan is a mixture of polysaccharides, which are viscous polymer electrolytes extracted from red algae (Rhodophyceae class), which is a natural seaweed, or Irish moss, and has been widely used as an emulsifier, a dispersant, a stabilizer, a swelling agent, a thickener, a binder, and an anti-crystallizer.

In addition, the carrageenan is a complex polysaccharide containing galactose and anhydro galactose as main components, and a sodium ester material, which is a carrageenan copolymer having a molecular weight (Mw) of 200,000 to 400,000, is used.

The lysine is one of the amino acids and may be used as an additive to food or feed. In the present invention, the lysine may be used to increase the external adaptability of microorganisms.

The potassium chloride is used to increase the solubility of the composition constituting the culture solution, the sodium citrate is used as an electrolyte chloride, the ethylene glycol is used to increase the nutrient absorption rate of microorganisms, the peptone is a nitrogen source that supplies protein and supplies protein necessary for the growth of microorganisms, the malt extract contains various minerals, the vitamin B group, and amino acids, which help the growth of microorganisms, and the pyridoxine hydrochloride may be used as a vitamin supplement to increase the activity of microorganisms.

Next, a microbial strain may be inoculated into the culture medium.

The inoculation may be performed by inoculating microbial strains consisting of a lactic acid bacteria strain, a Bacillus subtilis strain, and yeast into a culture medium, respectively. As the lactic acid bacteria, one or more selected from the group consisting of strains of the genus Lactobacillus, Lactococcus, Leuconostoc, Pediococcus, and Bifidobacterium may be used.

For example, the lactic acid bacteria may be a strain of the genus Lactobacillus, and the genus Lactobacillus strain may be one or more strains selected from the group consisting of Lactobacillus plantarum, Lactobacillus acidophilus, Lactobacillus bulgaricus, Lactobacillus sakei, Lactobacillus casei, and Lactobacillus brevis. Preferably, the lactic acid bacteria may be Lactobacillus plantarum.

In addition, the yeast is a microorganism used to make bread, beer, wine, and the like, and refers to a unicellular organism that is a group of molds or mushrooms but does not have hyphae and does not have photosynthetic performance or motility. Preferably, Saccharomyces cerevisiae may be used as the yeast.

In addition, the Bacillus subtilis may be one or more selected from the group consisting of Bacillus subtilis, Bacillus subtilis sp. zephyra, Bacillus Velezensis, Bacillus megaterium, Bacillus licheniformis, and Bacillus pumilus, and preferably, Bacillus Velezensis may be used as the Bacillus subtilis.

In addition, an inoculation amount of the microbial strain may have an initial bacterial count of 1×10⁶ to 1×10⁸ CFU/mL.

Next, after culturing microorganisms in a culture medium inoculated with the microbial strain, the cultured microorganisms may be mixed to produce a mixed culture solution.

Specifically, a mixed culture solution may be prepared by culturing the culture medium inoculated with lactic acid bacteria, Bacillus subtilis and yeast, respectively, at a temperature of 38 to 42° C. for 20 to 40 hours to prepare a lactic acid bacteria culture solution, a Bacillus subtilis culture solution, and a yeast culture solution and mixing the lactic acid bacteria culture solution, the Bacillus subtilis culture solution, and the yeast culture solution at a weight ratio of 1:1:1. Next, a microbial culture solution may be prepared by centrifuging the mixed culture solution to obtain a lower layer.

For example, the microbial culture solution may be prepared by centrifuging the mixed culture solution at 8,000×g to 12,000×g to obtain a lower layer solution. Since the configuration of centrifuging the mixed culture solution using a known centrifuge is a known technology, a detailed description thereof will be omitted for the convenience of explanation and clarity of the technical idea of the present invention.

Next, the fermented herbal medicine materials may be dried to produce a fermented dried herbal medicine material.

By drying the fermented herbal medicine materials to gradually remove moisture, the physical properties of the fermented herbal materials may be prevented from changing and the content of the active ingredient may be maximized. For example, the fermented dried herbal materials may be prepared by drying the fermented herbal materials at a temperature of 50 to 60° C. for 1 to 5 days.

Next, the fermented dried herbal materials may be extracted to produce a fermented herbal extract.

The fermented herbal extract may be prepared by extracting the fermented dried herbal materials using a known extraction method. For example, the extraction of the fermented dried herbal materials may utilize various known extraction methods such as a hot water extraction method, an organic solvent extraction method, an ultrasonic extraction method, a supercritical extraction method, and the like, and since the composition of the extraction is a known technology, a detailed description thereof will be omitted for the convenience of explanation and the clarity of the technical idea of the present invention.

The coenzyme Q10 is known to be involved in the synthesis of adenosine triphosphate (ATP) in mitochondria within cells. In general, human skin is exposed to a lot of environmental oxidative stress, which reduces the content of coenzyme Q10 in the skin and causes various problems such as photoaging. It is known that supplying coenzyme Q10 to the skin can prevent the decrease in content due to oxidative stress and, as a result, is effective in delaying aging.

The tocopherol acetate is an antioxidant that contributes to the first defense against lipid peroxidation components and can play a role in protecting cells from damage caused by oxidation.

The tocopherol acetate has an antioxidant action that scavenges reactive oxygen in vivo and inhibits the production of free radicals and lipid peroxides. The tocopherol acetate itself is unstable, so its derivatives are widely used, and in particular, vitamin acetate and vitamin palmitate are mainly used.

The butylene glycol is an aliphatic diol, also called 1,3-butanediol, and is a viscous liquid with almost no odor, a sweet taste, and hygroscopicity. It is known to have a moisturizing effect as it dissolves in water and ethanol and has excellent antibacterial properties and moderate wetting properties.

The vitamin C is called L-ascorbic acid (Asc), and it is known that vitamin C has an antioxidant function, prevents the oxidation of cholesterol, maintains blood vessels, and increases interferon production, and thus is effective in boosting immunity during initial infection with the influenza virus, preventing colds, and improving cold symptoms. In addition, vitamin C is effective in eliminating lead and mercury accumulated in the body.

The madecassoside component is a material isolated from Centella asiatica. The Centella asiatica is a small herbaceous plant belonging to the Umbelliferae family that grows wild in tropical and subtropical areas, ideally at altitudes of 600 to 800 meters above sea level. It is known to contain asiaticoside, madecassoside, asiatic acid, and madecassic acid. The madecassoside has a skin-soothing effect, helps strengthen the skin barrier, and aids in tissue recovery.

The olive liquid is a solubilizer extracted from olive oil and, unlike other emulsifiers, has its own moisturizing properties and is thus effective for atopic dermatitis and sensitive skin.

The sodium laureth sulfate is a known surfactant that may be easily purchased and used commercially by those skilled in the art.

The cocamide MIPA is mainly a mixture of coconut acid and isopropanolamide and has the properties of a surfactant, and can also perform functions such as a thickener, foaming agent, and binder.

The hydrolyzed collagen may be used for skin conditioning and may be a hydrolysate of animal and fish collagen obtained by acid, enzyme or other hydrolysis methods, and may be expected to have a moisturizing effect by promoting the regeneration of skin fibers, adjusting the pH of the skin, and activating metabolism.

The brewer's yeast extract is extracted from brewer's yeast, and the brewer's yeast is used in brewing beer and making alcoholic drinks and bread. Brewer's yeast is one of the yeasts needed to make beer. It is a non-fermentative yeast of the genus Saccharomyces that is separated after the fermentation of the wort is completed and the beer is filtered during the beer manufacturing process and contains about 45% protein and is high in vitamin B and phosphorus. In addition, the strain has the characteristics of producing high-concentration ethanol and being able to withstand high-concentration ethanol environments. Brewer's yeast includes a large amount of protein, nucleic acids, carbohydrates, minerals, and vitamins.

The brewer's yeast may be yeast that can be obtained during the beer fermentation process. The brewer's yeast may include one or more yeasts of the genus Saccharomyces selected from Saccharomyces cerevisiae, Saccharomyces carlsbergenesis, and Saccharomyces bayanus.

The acai berry extract is extracted from the acai berry, the fruit of the acai palm tree belonging to the palm family (Arecaceae) that grows in the Amazon rainforest region of northern Brazil, and is rich in amino acids such as aspartic acid and glutamic acid, oleic acid, unsaturated fatty acids, and minerals such as vitamin A, vitamin C, calcium, and iron. In particular, the skin of the acai berry contains functional components such as anthocyanin, a water-soluble pigment of the flavonoid family, and beta-sitosterol, a type of phytosterol, which are known to have the effect of removing harmful reactive oxygen.

The emollient oil may be used to provide a moisturizing effect to the skin and to dissolve a useful physiologically active component. For example, the emollient oil may be one or more oils selected from the group consisting of squalane, trioctanoate, cetearyl octanoate cyclomethicone, and dimethicone.

The Artemisiae capillaris extract is extracted from Artemisiae capillaris and refers to a dried medicinal material derived from the aerial part of Artemisia capillaris Thunberg of the Asteraceae family. The Artemisiae capillaris is used for jaundice caused by damp heat, i.e., fever, yellowing of the whole body, and red urine and low urine output due to acute hepatitis.

In addition, the Artemisiae capillaris is also used for chronic hepatitis, cirrhosis, liver cancer, cholecystitis, gallstones, skin diseases such as eczema, scabies, ringworm, and rubella, and symptoms of fever and eczematous rash. The pharmacological effects of Artemisiae capillaris have been reported to include bile secretion promotion, antipyretic, diuretic, antimicrobial, and experimental ascites cancer cell inhibition effects.

The Allium hookeri extract is extracted from Allium hookeri and is also generally called three greens because it has a sweet, bitter, and spicy taste, and is also called ginseng greens because it tastes like ginseng, and the ginseng greens are a crop that reproduces vegetatively like garlic.

Dietary sulfur, which is the main component of the Allium hookeri, contains 60 times more saponin than wild ginseng, and is rich in nitrogen and fiber along with minerals such as calcium, iron, zinc, and manganese, so it has various effects such as formation of bones and teeth, blood production, blood purification, increased vitality, body balance regulation, prevention of hypertension, arteriosclerosis, constipation, and bowel movement promotion.

The role of the dietary sulfur is closely related to the prevention and treatment of alpha- and gamma-psychotropic diseases (dementia and mental retardation), beta-cancer or chronic diseases (diabetes, hypertension, and hypotension), delta-skin diseases (agent orange, psoriasis, and atopic allergies), liver function improvement and detoxification, such as strengthening the lambda and mu sulfur-immune system, and while ginseng, red ginseng, wild ginseng, and sun ginseng only contain beta sulfur, it is known that Allium hookeri contains all six components.

The grapefruit seed extract is extracted from the seeds of grapefruit, and the grapefruit seeds contain ascorbic acid, sterol, dehydroascorbic acid, tocopherol, palmitic acid, and the like as main components, and various effective components are known to inhibit the growth of microorganisms by weakening the enzyme activity and permeability related to the cell membrane of microorganisms and inhibiting cellular respiration.

In particular, the grapefruit seeds contain naringin, which is a bioflavonoid, and citral, as their main components, so they not only have excellent antibacterial and antioxidant effects, but also have excellent antibacterial effects against gram-positive and gram-negative bacteria as well as fungi.

The sodium alginate is a white-light yellow fiber, grain, granule, or powder with the chemical formula (C₆H₇O₆Na)ₙ, and is a bio-derived material with almost no odor and no taste, and is used as a stabilizer for ice cream, an adhesive for ketchup, mayonnaise, and sauce, a clarifying agent for sake, a gelling agent, an emulsifier, and a thickener to increase the adhesiveness and viscosity of food, promote emulsification stability, and improve the physical properties and texture of food.

The sodium alginate is a polymeric material and present in various grades through a purification process, and when dissolved in water, it becomes viscous and the pH of a 1% aqueous solution is 6 to 8, which means it has almost neutral properties. Sodium alginate has the property of hardening when it comes into contact with sodium chloride (NaCl) or under low pH conditions. Due to these properties, sodium alginate has been used as a thickener, and the like.

The sodium alginate may be prepared by extraction from the Sargassum horneri, and for example, the extraction of the sodium alginate is performed according to the method of Calumpong et al (1999). Specifically, the sodium alginate may be prepared by adding 800 mL of 2% formaldehyde to 25 g of Sargassum horneri to perform a first pretreatment at room temperature for 24 hours, washing the Sargassum horneri with water, performing a second pretreatment in 800 mL of 0.2 M hydrochloric acid (HCl) for 24 hours, immersing the Sargassum horneri that has undergone the pretreatment process in 1 L of 2% sodium carbonate (Na₂CO₃) to extract a solution at a temperature of 60 to 80° C. for 1 to 3 hours, centrifuging the extracted solution at 3200 rpm for 25 minutes, precipitating it in 3 times the volume of 95% ethanol, and finally drying it at 60° C. for 50 hours,

The Moutan cortex radicis extract is prepared by extracting Moutan cortex radicis. Moutan cortex radicis is an important herbal medicine that has been used for a long time and is a deciduous small shrub belonging to the Ranunculaceae family, which is called the ‘peony root bark’. Its main components are paeonoside (paeonol glucoside), paeonolide (paeonol-rhamnoglucoside), and paeonol, and the paeonoside decomposes during storage to produce sugar and paeonol. It was found that the paeonol has antibacterial properties and inhibits the proliferation of Escherichia coli, Staphylococcus aureus, Streptococcus pneumoniae, and Bacillus subtilis diluted 1,500 to 2,000-fold in vitro. Moutan cortex radicis also contains components such as paeoniflorin, oxypaeoniflorin, benzoylpaeoniflorin, paeonolide tannin, procyanidin B1, benzoyloxypaeoniflorin, paeonin, astragalin, and pelargonin. Histochemistry confirmed that these components are present in the xylem as well as the rhizodermis. Paeoniflorin inhibits platelet aggregation and reduces fibrinogen. Benzoylpaeoniflorin and benzoyloxypaeoniflorin have the effect of inhibiting histamine release from mast cells, and paeonolide tannin has an antiviral effect. It is thought that the anti-inflammatory effect is due to the monoterpene glycoside component. Since Moutan cortex radicis is cool in nature, it is used in oriental medicine as an anti-inflammatory and blood-thinning medicine. Its medicinal effects are used for inflammation of the blood vessels of the lower abdominal organs, pain due to congestion, fever, suppuration, bleeding, and the like. In the gynecological field, it has anti-inflammatory, analgesic, and sedative effects for irregular menstruation, inflammation of the uterus and appendages, congestion, and traction pain, and it is also applied to hemorrhoids and appendicitis.

The pine needle shoot extract is prepared by extracting pine needle shoots. The pine needle shoots are the leaves and shoots of pine trees (Pinus densiflora Siebold et Zuccarini), which have a long history with people in Northeast Asia, including Korea, China, and Japan. Fresh pine needles contain large amounts of ascorbic acid, vitamins A, B, and K, bitter materials, flavonoids, anthocyanins, 7 to 12% resin, about 5% tannin, carbohydrates, and essential oils.

In addition, the entire pine tree contains components that promote metabolism and excrete human waste from the body, such as alcohol and ester, phenol compounds, quinine, turpentine, vitamins A and C, and chlorotyl as main components, as well as glycoginine and apietic acid. It is also rich in iron, and 24 types of amino acids are contained in red pine leaves, and 19 types of amino acids that make up proteins have also been confirmed.

The pine needles are the source and raw material of vitamin C, and since they have a much darker green color, abundant carotene can be consumed. In the case of pine needles, the pharmacological value may refer to the general effects of terpenes, phenolic compounds, and tannins. In addition, they are good for hypertension, stroke, diabetes, dementia, and anti-aging, anti-cancer effects, the liver and stomach, constipation and anemia, chronic alcoholism and hangover relief, nicotine removal, skin beauty, constitution improvement, stamina of dieting men, various stresses, excessive smoking, and students and test takers who want to clear their heads and study especially when their heads are heavy and their eyes are blurry due to drinking.

Salicornia herbacea extract is prepared by extracting Salicornia herbacea. The Salicornia herbacea (Salicornia spp.) is an annual grass of the glasswort family and is a representative edible and medicinal halophyte native to Korea. The Salicornia herbacea has been used as a folk medicine for a long time to treat vision loss, indigestion, gastrointestinal diseases, hepatitis, kidney diseases, and the like, and its antioxidant, antidiabetic, anticancer, antihypertensive, antihyperlipidemic, and skin whitening effects have been scientifically proven.

The jojoba seed oil is extracted by pressing the seeds of the jojoba plant that grows in desert areas, and is an oil extracted in the form of liquid wax. The jojoba seed oil is well absorbed into the skin and delivers moisture deep into the skin without clogging pores, making it effective in maintaining skin balance. The jojoba seed oil is good for dry or aged skin and has an excellent moisturizing effect that helps prevent moisture loss by forming a moisture film on the skin.

Hereinafter, the cosmetic composition for skin improvement containing low-molecular-weight collagen, collagen water, or a collagen extract according to one embodiment of the technical idea of the present invention will be described in more detail with reference to examples.

### Example 1

A cosmetic was prepared by mixing 0.2 wt % of low-molecular-weight collagen powder, 1 wt % of collagen water, 0.5 wt % of collagen extract, 0.5 wt % of caffeic acid, 0.3 wt % of soybean-derived lysolecithin, 1 wt % of fermented herbal extract, 0.1 wt % of coenzyme Q10, 0.2 wt % of tocopherol acetate, 1 wt % of butylene glycol, 0.2 wt % of stearyltrimethylammonium chloride, 0.1 wt % of L-aspartic acid, 0.1 wt % of sorbitol, 1 wt % of stearyl alcohol, 1 wt % of cyclopentasiloxane, 0.3 wt % of vitamin C, 0.1 wt % of madecassoside, 0.05 wt % of olive liquid, 1 wt % of sodium laureth sulfate, 0.3 wt % of cocamide MIPA, 0.1 wt % of beer yeast extract, 0.05 wt % of acai berry extract, 0.1 wt % of emollient oil, 0.05 wt % of Artemisiae capillaris extract, 0.03 wt % of Allium hookeri extract, 0.03 wt % of grapefruit seed extract, 0.03 wt % of sodium alginate, 0.03 wt % of Moutan cortex radicis extract, 0.03 wt % of pine needle shoot extract, 0.06 wt % of panthenol, 0.03 wt % of salicylic acid, 0.1 wt % of polyquaternium-7, 0.1 wt % of niacinamide, 0.2 wt % of jojoba seed oil, 0.5 wt % of behenyl alcohol, 1 wt % of myristyl alcohol, 0.1 wt % of cyclodextrin, 0.05 wt % of fragrance, and the balance as deionized water.

### Example 2

A cosmetic was prepared by mixing 0.25 wt % of low-molecular-weight collagen powder, 0.7 wt % of collagen water, 0.9 wt % of collagen extract, 0.3 wt % of caffeic acid, 0.4 wt % of soybean-derived lysolecithin, 0.8 wt % of fermented herbal extract, 0.11 wt % of coenzyme Q10, 0.15 wt % of tocopherol acetate, 1.3 wt % of butylene glycol, 0.15 wt % of stearyltrimethylammonium chloride, 0.09 wt % of L-aspartic acid, 0.13 wt % of sorbitol, 0.7 wt % of stearyl alcohol, 1.1 wt % of cyclopentasiloxane, 0.2 wt % of vitamin C, 0.13 wt % of madecassoside, 0.03 wt % of olive liquid, 1.1 wt % of sodium laureth sulfate, 0.2 wt % of cocamide MIPA, 0.14 wt % of beer yeast extract, 0.03 wt % of acai berry extract, 0.11 wt % of emollient oil, 0.03 wt % of Artemisiae capillaris extract, 0.04 wt % of Allium hookeri extract, 0.02 wt % of grapefruit seed extract, 0.04 wt % of sodium alginate, 0.02 wt % of Moutan cortex radicis extract, 0.04 wt % of pine needle shoot extract, 0.05 wt % of panthenol, 0.04 wt % of salicylic acid, 0.09 wt % of polyquaternium-7, 0.12 wt % of niacinamide, 0.15 wt % of jojoba seed oil, 0.9 wt % of behenyl alcohol, 0.7 wt % of myristyl alcohol, 0.12 wt % of cyclodextrin, 0.05 wt % of fragrance, and the balance as deionized water.

### Example 3

A cosmetic was prepared by mixing 0.15 wt % of low-molecular-weight collagen powder, 1.4 wt % of collagen water, 0.3 wt % of collagen extract, 0.8 wt % of caffeic acid, 0.4 wt % of soybean-derived lysolecithin, 1.3 wt % of fermented herbal extract, 0.09 wt % of coenzyme Q10, 0.25 wt % of tocopherol acetate, 0.7 wt % of butylene glycol, 0.25 wt % of stearyltrimethylammonium chloride, 0.11 wt % of L-aspartic acid, 0.09 wt % of sorbitol, 1.3 wt % of stearyl alcohol, 0.9 wt % of cyclopentasiloxane, 0.4 wt % of vitamin C, 0.07 wt % of madecassoside, 0.08 wt % of olive liquid, 0.9 wt % of sodium laureth sulfate, 0.4 wt % of cocamide MIPA, 0.07 wt % of beer yeast extract, 0.08 wt % of acai berry extract, 0.09 wt % of emollient oil, 0.08 wt % of Artemisiae capillaris extract, 0.02 wt % of Allium hookeri extract, 0.04 wt % of grapefruit seed extract, 0.02 wt % of sodium alginate, 0.04 wt % of Moutan cortex radicis extract, 0.02 wt % of pine needle shoot extract, 0.07 wt % of panthenol, 0.02 wt % of salicylic acid, 0.11 wt % of polyquaternium-7, 0.08 wt % of niacinamide, 0.25 wt % of jojoba seed oil, 0.3 wt % of behenyl alcohol, 1.3 wt % of myristyl alcohol, 0.09 wt % of cyclodextrin, 0.05 wt % of fragrance, and the balance as deionized water.

### Comparative Example 1

A cosmetic was prepared by mixing 0.5 wt % of coenzyme Q10, 0.5 wt % of tocopherol acetate, 1.3 wt % of butylene glycol, 0.2 wt % of stearyltrimethylammonium chloride, 0.5 wt % of L-aspartic acid, 0.2 wt % of sorbitol, 1 wt % of stearyl alcohol, 1.5 wt % of cyclopentasiloxane, 0.8 wt % of vitamin C, 0.3 wt % of madecassoside, 0.08 wt % of olive liquid, 1.2 wt % of sodium laureth sulfate, 0.5 wt % of cocamide MIPA, 0.3 wt % of beer yeast extract, 0.1 wt % of acai berry extract, 0.3 wt % of emollient oil, 0.05 wt % of grapefruit seed extract, 0.03 wt % of Moutan cortex radicis extract, 0.1 wt % of panthenol, 0.03 wt % of salicylic acid, 0.15 wt % of polyquaternium-7, 0.18 wt % of niacinamide, 0.8 wt % of jojoba seed oil, 0.9 wt % of behenyl alcohol, 3 wt % of myristyl alcohol, 0.5 wt % of cyclodextrin, 0.05 wt % of fragrance, and the balance as deionized water.

### Comparative Example 2

A cosmetic was prepared by mixing 1.5 wt % of collagen extract, 0.5 wt % of caffeic acid, 0.5 wt % of coenzyme Q10, 0.6 wt % of tocopherol acetate, 1.5 wt % of butylene glycol, 0.5 wt % of sorbitol, 1.5 wt % of stearyl alcohol, 2 wt % of cyclopentasiloxane, 0.3 wt % of vitamin C, 0.5 wt % of madecassoside, 0.05 wt % of olive liquid, 0.2 wt % of acai berry extract, 0.1 wt % of emollient oil, 0.05 wt % of Artemisiae capillaris extract, 0.1 wt % of Moutan cortex radicis extract, 0.3 wt % of pine needle shoot extract, 0.08 wt % of panthenol, 0.1 wt % of salicylic acid, 0.15 wt % of polyquaternium-7, 0.3 wt % of niacinamide, 0.5 wt % of jojoba seed oil, 1.5 wt % of behenyl alcohol, 1.3 wt % of myristyl alcohol, 0.5 wt % of cyclodextrin, 0.05 wt % of fragrance, and the balance as deionized water.

### 1. Skin Brightness Improvement Evaluation

The skin brightness improvement effect of the cosmetics according to Examples 1, 2, and 3 and the cosmetics according to Comparative Examples 1 and 2 was evaluated.

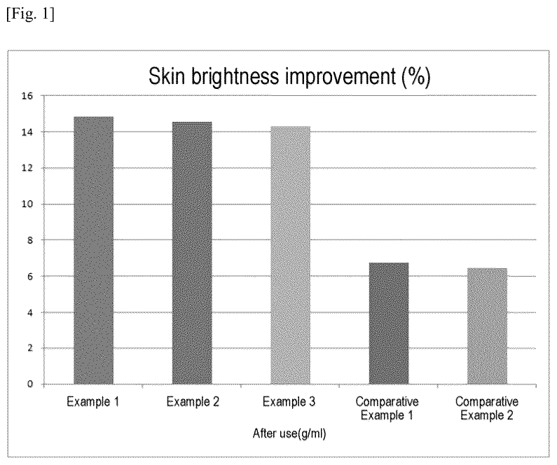

FIG. 1 is a graph showing the skin brightness improvement effect of the cosmetics according to Examples 1, 2, and 3 and the cosmetics according to Comparative Examples 1 and 2.

The skin brightness improvement effect was confirmed by measuring the skin brightness of 40 women selected from among 20 to 40 years of age before use using Chromameter CR-400, and then measuring the skin brightness again after using the cosmetics according to Examples 1, 2, and 3 and the cosmetics according to Comparative Examples 1 and 2 individually.

The above measurement results are shown in Table 1 below and FIG. 1, and the measurement values are expressed as the average of three measurements excluding the maximum and minimum values of L*.

TABLE 1
[Skin brightness (L* Arbitrary unit)]

| | | | | Comparative | Comparative |
|---|---|---|---|---|---|
| Classification | Example 1 | Example 2 | Example 3 | Example 1 | Example 2 |
| Before use | 61.58 | 60.73 | 62.43 | 61.21 | 62.28 |
| After use | 70.71 | 69.58 | 71.35 | 65.34 | 66.31 |
| Increase rate | 14.83 | 14.57 | 14.29 | 6.75 | 6.47 |
| (%) | | | | | |

Referring to the above Table 1 and FIG. 1, it can be confirmed that the cosmetics according to Examples 1, 2, and 3 significantly improved skin brightness compared to the cosmetics according to Comparative Examples 1 and 2.

### 2. Evaluation of Skin Wrinkle Improvement Effect

The skin wrinkle improvement effect of the cosmetics according to Examples 1, 2, and 3 and the cosmetics according to Comparative Examples 1 and 2 was evaluated.

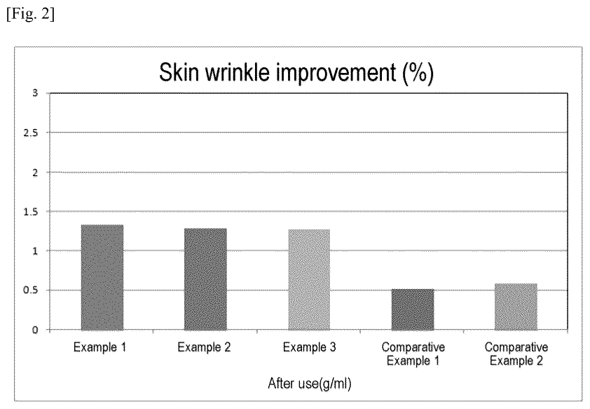

FIG. 2 is a graph showing the skin wrinkle improvement effect of the cosmetics according to Examples 1, 2, and 3 and the cosmetics according to Comparative Examples 1 and 2.

The above skin wrinkle improvement effect was measured on 50 women aged 30 or older.

The cosmetics according to Examples 1, 2, and 3 and the cosmetics according to Comparative Examples 1 and 2 were applied twice a day (morning and evening) around the eyes for 10 weeks, and the wrinkle improvement effect on the skin of the subjects was measured using the ANTERA 3D skin wrinkle improvement measuring instrument.

The test results were recorded as the A1 (A1 (10 weeks)-A1 (0 weeks)) value of the measuring instrument. A1 value represents the properties of skin wrinkle viscoelasticity, and the results are as shown in Table 2 below and FIG. 2.

TABLE 2

| Classification | Skin wrinkle improvement effect |
|---|---|
| Example 1 | 1.33 |
| Example 2 | 1.28 |
| Example 3 | 1.27 |
| Comparative Example 1 | 0.51 |
| Comparative Example 2 | 0.58 |

Referring to Table 2 and FIG. 2, it can be confirmed that the cosmetics according to Examples 1, 2, and 3 have a significantly superior effect on improving skin wrinkles compared to the cosmetics according to Comparative Examples 1 and 2.

### 3. Whitening Improvement Experiment

Melanin tyrosinase is the main enzyme that produces melanin pigment. In order to confirm the whitening effect of the cosmetic according to Example 1, the amount of dihydroxyphenylalanine (DOPA) or dopachrome produced by tyrosinase, the main enzyme of melanin synthesis, was compared to measure the enzyme activity inhibition ability of the test material.

The test method involved using tyrosine as a substrate, adding it to an enzyme reaction solution, reacting it for a certain period of time, and then measuring the absorbance at 475 nm, which is the absorption wavelength of the reaction product, dopachrome.

500 μl of L-tyrosine (0.3 mg/ml) and 450 μl of each of the cosmetics according to Example 1 and Comparative Example 1 were added to 500 μl of a 0.1 M potassium phosphate buffer solution (pH 6.8), allowed to react in a 37° C. water bath for 10 minutes, and then quickly placed in a refrigerator for 5 minutes to stop the reaction. 50 μl of tyrosinase (1,250 units/ml) was added and incubated for 10 minutes, and then the absorbance was measured at 475 nm. For the control group, 450 μl of a sample solvent was added instead of the test solution. For the color correction blank, 50 μl of the potassium phosphate buffer solution (pH 6.8, 0.1 M) was added instead of tyrosinase, and a sample with the same concentration as the experimental group was added. Tyrosinase inhibition activity was calculated using the following Mathematical Formula 1 and is shown in Table 3.

$$\% Inhibition = [ ( A - B ) / A ] \times 100 \qquad [ Mathematical Formula 1 ]$$

A: Absorbance after reaction of the reaction solution (control) without test sample (inhibitor)
B: Absorbance after reaction of the reaction solution with test sample

TABLE 3

| Classification | Tyrosinase inhibition activity (%) |
|---|---|
| Example 1 | 36.87 |
| Comparative Example 1 | 12.33 |
| Positive control (Arbutin) | 40.52 |

Referring to Table 3, it was confirmed that the cosmetic according to Example 1 showed a tyrosinase inhibition rate of 36.87%, while the cosmetic according to Comparative Example 1 showed a tyrosinase inhibition rate of 12.33%.

In addition, the cosmetic according to Example 1 showed a slightly lower inhibition rate than the control, arbutin. Therefore, the cosmetic according to Example 1 can be said to be effective in inhibiting tyrosinase.

### 4. Moisturizing Effect Measurement

A capacitance measurement method using Corneometer CM820 (Courage, Germany) was used, and the moisturizing effect was comparatively measured using the cosmetic according to Example 1 and the cosmetic according to Comparative Example 1.

The subjects were 20 women aged 20 to 50, and the inner side of the lower arm was allowed to adapt to the test for 20 minutes in a constant temperature and humidity room immediately before the test began, and the skin moisture was maintained at a constant level. Next, the initial moisture retention amount of each sample application area was measured using Corneometer C820, and then the cosmetic according to Example 1 and the cosmetic according to Comparative Example 1 were applied.

The moisture increase/decrease rate was first measured 20 minutes after application, and then measured again 2 hours later. Each measurement area was repeatedly measured three times, and the average value was used to calculate the skin moisture increase/decrease rate according to Mathematical Formula 2, which is shown in Table 4.

Moisture increase/decrease rate (%)=(Tdi−Tdo)−(Ntdi−NTdo)/((Ntdi−Ntdo)+Tdo)*100 [Mathematical Formula 2]
Tdi: n^{th} measurement value of sample application area
Tdo: Measurement value before sample application
Ntdi: n^{th} measurement value for area where no sample was applied
NTdo: Pre-application measurement value for area where no sample was applied

TABLE 4

| Classification | Example 1 | Comparative Example 1 |
|---|---|---|
| 20 min | 37.84 | 18.27 |
| 120 min | 29.57 | 6.33 |

Referring to Table 4, it can be confirmed that the cosmetic according to Example 1 has a superior moisturizing effect not only initially but also over time compared to the cosmetic according to Comparative Example 1.

### 4. Formulation Stability Test

A stability test was performed on the cosmetics prepared according to Examples 1, 2, and 3 above.

The observation period was the same for one week, and stability was observed at various temperatures, and then the external appearance, discoloration, and odor were checked, and the results are shown in Table 5.

TABLE 5

| | Example 1 | | | Example 2 | | | Example 3 | | |
|---|---|---|---|---|---|---|---|---|---|
| Temper- | Appear- | Discol- | Bad | Appear- | Discol- | Bad | Appear- | Discol- | Bad |
| ature | ance | oration | smell | ance | oration | smell | ance | oration | smell |
| 0 | 0 | 0 | 0 | 0 | 0 | 0 | 0 | 0 | 0 |
| 40 | 0 | 0 | 0 | 0 | 0 | 0 | 0 | 0 | 0 |
| −20 | 0 | 0 | 0 | 0 | 0 | 0 | 0 | 0 | 0 |

(∘: Good, Δ: Minor denaturation, x: Significant denaturation)

Referring to Table 5, it can be confirmed that the cosmetics prepared according to Examples 1, 2, and 3 have good formulation stability.

Although one preferred embodiment of the present invention has been described above with reference to the accompanying drawings, those of ordinary skill in the art to which the present disclosure pertains should understand that the present disclosure may be embodied in other specific forms without changing the technical spirit or essential features thereof. Therefore, it should be understood that the embodiments described above are illustrative in all respects and not restrictive.

What is claimed is:

1. A cosmetic composition for skin improvement, comprising one or more collagens selected from low-molecular-weight collagen powder, collagen water, or a collagen extract,
wherein the collagen powder is a collagen powder having a molecular weight of 200 to 300 daltons (Da).

2. The cosmetic composition of claim 1, wherein the low-molecular-weight collagen powder is a collagen powder which is prepared by preparing one or more collagens selected from the group consisting of collagen extracted from fish and fish scales, chicken foot collagen, beef foot collagen, and pork foot collagen, sterilizing the collagen, mixing 80 to 120 parts by weight of the sterilized collagen, 20 to 40 parts by weight of a proteolytic enzyme, and 150 to 250 parts by weight of purified water to produce a collagen enzyme mixture, heating the collagen enzyme mixture in a double boiler at a temperature of 57 to 635° C. for 1 to 5 hours to produce a collagen enzyme hydrolyzate, immersing the collagen enzyme hydrolyzate in sodium hypochlorite for 10 to 60 minutes to decolorize and deodorize the same, concentrating the decolorized and deodorized collagen enzyme hydrolyzate to produce a collagen concentrate, and spray drying the collagen concentrate at a temperature of 160 to 180° C.,
the collagen water is collagen water which is prepared by preparing one or more collagens selected from the group consisting of collagen extracted from fish and fish scales, chicken foot collagen, beef foot collagen, and pork foot collagen, sterilizing the collagen, adding 10- to 30-fold (v/w) distilled water to the sterilized collagen, heating the mixture at 60 to 80° C. for 10 to 50 minutes, then distilling and extracting the mixture while heating at 100° C. to recover the distillate at 30 to 40% of the total weight of the mixture and produce a collagen distillation extract, and filtering the collagen distillation extract through a filter, and
the collagen extract is a collagen extract which is prepared by preparing one or more collagens selected from the group consisting of collagen extracted from fish and fish scales, chicken foot collagen, beef foot collagen, and pork foot collagen, sterilizing the collagen, mixing 80 to 120 parts by weight of purified water based on 100 parts by weight of the total content of the sterilized collagen, heating the mixture at a temperature of 90 to 100° C. for 1 to 3 hours and performing hot water extraction to produce a collagen hot water extract, and filtering the collagen hot water extract through a filter.

3. The cosmetic composition of claim 2, wherein, in addition to the low-molecular-weight collagen powder, collagen water, and collagen extract, the cosmetic composition includes caffeic acid, soybean-derived lysolecithin, a fermented herbal extract, coenzyme Q10, tocopherol acetate, butylene glycol, stearyl trimethyl ammonium chloride, L-aspartic acid, sorbitol, stearyl alcohol, cyclopentasiloxane, vitamin C, madecassoside, olive liquid, sodium laureth sulfate, cocamide MIPA, brewer's yeast extract, acai berry extract, emollient oil, Artemisiae capillaris extract, Allium hookeri extract, grapefruit seed extract, sodium alginate, Moutan cortex radicis extract, pine needle shoot extract, panthenol, salicylic acid, polyquaternium-7, niacinamide, jojoba seed oil, behenyl alcohol, myristyl alcohol, cyclodextrin, a fragrance, and the balance as deionized water.

4. The cosmetic composition of claim 3, wherein the cosmetic composition includes 0.1 to 0.3 wt % of the low-molecular-weight collagen powder, 0.5 to 1.5 wt % of the collagen water, 0.1 to 1 wt % of the collagen extract, 0.1 to 1 wt % of caffeic acid, 0.1 to 0.5 wt % of soybean-derived lysolecithin, 0.5 to 1.5 wt % of fermented herbal extract, 0.08 to 0.12 wt % of coenzyme Q10, 0.1 to 0.3 wt % of tocopherol acetate, 0.5 to 1.5 wt % of butylene glycol, 0.1 to 0.3 wt % of stearyltrimethylammonium chloride, 0.08 to 0.12 wt % of L-aspartic acid, 0.05 to 0.15 wt % of sorbitol, 0.5 to 1.5 wt % of stearyl alcohol, 0.8 to 1.2 wt % of cyclopentasiloxane, 0.1 to 0.5 wt % of vitamin C, 0.05 to 0.15 wt % of madecassoside, 0.01 to 0.1 wt % of olive liquid, 0.8 to 1.2 wt % of sodium laureth sulfate, 0.1 to 0.5 wt % of cocamide MIPA, 0.05 to 0.15 wt % of brewer's yeast extract, 0.01 to 0.1 wt % of acai berry extract, 0.08 to 0.12 wt % of emollient oil, 0.01 to 0.1 wt % of Artemisiae capillaris extract, 0.01 to 0.05 wt % of Allium hookeri extract, 0.01 to 0.05 wt % of grapefruit seed extract, 0.01 wt % to 0.05 wt % of sodium alginate, 0.01 to 0.05 wt % of Moutan cortex radicis extract, 0.01 to 0.05 wt % of pine needle shoot extract, 0.04 to 0.08 wt % of panthenol, 0.01 to 0.05 wt % of salicylic acid, 0.08 to 0.12 wt % of polyquaternium-7, 0.07 to 0.13 wt % of niacinamide, 0.1 to 0.3 wt % of jojoba seed oil, 0.1 to 1 wt % of behenyl alcohol, 0.5 to 1.5 wt % of myristyl alcohol, 0.07 to 0.13 wt % of cyclodextrin, 0.01 to 0.1 wt % of fragrance, and the balance as deionized water.

5. A method for skin improvement, comprising administering the composition according to claim 1 to a skin of a subject in need thereof.
